(19) United States
(12) Patent Application Publication
(10) Pub. No.: US 2003/0186891 A1
(43) Pub. Date: Oct. 2, 2003

(54) Compositions and methods for treatment of vitamin D deficiency

(76) Inventors: Toshio Okano, Kobe (JP); Naoko Tsugawa, Kobe (JP); Kimie Nakagawa, Kobe (JP); Russell W. Blacher, Castro Valley, CA (US); Yoshinari Kumagai, Alameda, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/360,202

(22) Filed: Feb. 7, 2003

Related U.S. Application Data
(60) Provisional application No. 60/355,548, filed on Feb. 8, 2002.

(51) Int. Cl. A61K 38/10; C07K 7/08

(52) U.S. Cl. 514/14; 530/326

(57) ABSTRACT

The present invention provides peptides which are characterized by having a biological activity that increases 25-hydroxyvitamin D3 1α-hydroxylase activity in a cell, thereby increasing calcitriol (active vitamin D) levels. The peptides have a sequence related to the contiguous sequence defined by residues 242 to 264 in the naturally occurring matrix extracellular phosphoglycoprotein (PHEX). Methods of modulating 25-hydroxyvitamin 1α-hydroxylase gene expression and calcitriol levels using the subject peptides are also provided. Also provided are kits for practicing the subject methods. The subject compositions and methods find use in a variety of application, including the treatment of vitamin D-related disorders, such as Paget's Disease, rickets, osteoporosis, renal osteodystrophy, and psoriasis.

### CROSS REFERENCE TO RELATED APPLICATIONS

This application claims priority to U.S. Provisional Patent Application Serial No. 60/355,548 filed Feb. 8, 2002; the disclosures of which application is herein incorporated by reference.

### INTRODUCTION

1. Technical Field

The present invention relates to peptides that may be used to manipulate vitamin D metabolism. More specifically, the present invention relates to dentonin peptides that stimulate increase 25-hydroxyvitamin D3 1α-hydroxylase activity to produce calcitriol, the active form of vitamin D.

2. Background of the Invention

Vitamin D is a hormone that plays an active role in the maintenance of calcium and phosphate balance and bone mineralization. Vitamin D deficiency causes rickets and osteomalacia in both adults and children. Both conditions are characterized by failure of calcification of osteoid, which is the matrix of bone, leading to weak and malformed bones. The active form of vitamin D, 1α,25-dihydroxyvitamin D3 (otherwise known as calcitriol), is made from inactive 25-monohydroxyvitamin D3 through a hydroxylation reaction performed by a 1α-hydroxylase (1α-OHase), which places a hydroxyl group on the 1α position of the carbon chain of the molecule. Hydroxylation of the precursor 25-monohydroxyvitamin D3 by a 24-hydroxylase (24-OHase) causes the pathway to “bypass” the active form of the compound. Hydroxylation of calcitriol by the 24-hydroxylase inactivates calcitriol, and initiates its further metabolism.

A large number of diseases have been associated with alterations in vitamin D metabolism. Familial diseases resulting in disorders of vitamin D metabolism have been identified as causes for rickets, including X-linked vitamin D resistant hypophosphatemic rickets (XLH or HYP), hereditary hypercalciuria with hypophosphatemic rickets (HHRH), Dent's disease including certain types of renal Fanconi syndrome, renal 1 alpha-hydroxylase deficiency (VDDR I), defects in 1,25-dihydroxy vitamin D3 receptor (end organ resistance, VDDR II), autosomal dominant rickets (ADR), and McCune-Albright Syndrome (MAS). Diseases that show rickets-like symptoms such as oncogenic hypophosphatemic osteomalacia (OHO) are also known. In addition to the classical diseases resulting from disorders of vitamin D metabolism, it is thought that several other diseases can be treated by manipulating vitamin D metabolism and increasing calcitriol levels, such as psoriasis and cancer (Abe et al, P.N.A.S. (1981), 78, 4990-4994), immunological disorders (Muller et al, Immunol. Lett. (1988) 17, 361-366), hypertension (Lind et al Acta Med Scand. (1987) 222, 423-427), diabetes mellitus (Inomata et al Bone Miner. (1986) 1, 187-192), alopecia (Editorial, Lancet (1989) i, p. 478), acne (Malloy, V. L. et al., Tricontinental Meeting for Investigative Dermatology, Washington, 1989), osteoporosis (Bikle Endocr. Rev. Monogr. (1995) 4, 77-83), neurodegenerative disorders (Carswell Exp. Neurol. (1993), 124, 36-42) and several other disease (see generally U.S. Pat. No. 6,329,357).

Currently, there is a need for an effective treatment for disorders of vitamin D metabolism, and a need to increase cellular and circulating calcitriol levels for diseases for which an increase would be therapeutic. For osteopenic diseases such as osteoporosis, therapeutic agents such as estrogen, calcitriol, vitamin D, fluoride, Iprifravon, bisphosphonates, parathyroid hormone and a few others have failed to provide a satisfactory means of treatment (Gennari et al., Drug Saf. (1994) 11(3):179-95; Martin et al., Am J Kidney Dis (2001) 38(6):1430-6). A significant problem with these treatments, for example as with exogenously administered vitamin D analogs, is that they increase intestinal calcium absorption and renal calcium reabsorption, leading to hypercalcemia. Injectable parathyroid hormone was recently developed as a novel skeletal anabolic agent. However, it needs to be administered to patients with certain intervals as continuous exposure to the drug accelerates bone resorption. Accordingly, the use of this drug requires a special care by the medical professionals. Another significant problem with the agents described above is that they are unsuitable for oral administration in most cases, and thus, must be given parenterally. Since bone disorders are often chronic and require long-term therapy, it is important that therapeutic agents be suitable for oral administration.

The regulation of vitamin D metabolism is beginning to be understood. Recently a gene, Phex, has been cloned and characterized that is defective in patients with X-linked hypophosphatemic rickets (Rowe, Hum. Mol. Genet. 6 (1997), 539-549). The Phex gene encodes is a type II glycoprotein and a member of a family (M13), of Zn metalloendopeptidases named PHEX. PHEX is proposed to function by processing a factor that plays a role in phosphate homeostasis and skeletal mineralization (Rowe, Exp. Nephrol. 5 (1997), 355-363). In diseased states such as familial rickets, defective PHEX results in uncleaved phosphatonin that would result in down regulation of the sodium dependent phosphate cotransporter, downregulation of 1α-hydroxylase (1α-OHase), and upregulation of renal mitochondrial 24-hydroxylase (24-OHase). The effects of PHEX on expression of genes in vitamin D metabolism are not understood.

A significant need exists for therapeutic agents that can prevent or treat diseases by altering in vitamin D metabolism. In particular, agents that can selectively increase the expression of the 1α-OHase over the 24-OHase are of interest as effective agents because they would selectively increase the levels of endogenous active 1,25-dihydroxyvitamin D3 over the inactive forms containing hydroxy-groups at the 24 position of the carbon chain. Such agents could be used as a treatment for diseases related to vitamin D, such as Paget's Disease, rickets, osteoporosis, renal osteodystrophy, psoriasis, and so on.

References of interest include: Yoshida et al., J Am Soc Nephrol. 2002 13:1455-63; Kato et al, Horm Res. 2002 57:73-8; Yoshida et al., Endocrinology. 2002 143:683-9; Miller et al., Best Pract Res Clin Endocrinol Metab. 2001 15:95-109; Zehnder et al., J Clin Endocrinol Metab. 2001 86:888-94; Zoidis et al., Mol Cell Endocrinol. 2000 168(1-2):41-51; Hewison et al., J Mol Endocrinol. 2000 25:141-8; Kimmel-Jehan et al., Biochim Biophys Acta. 2000 1475:109-13; Eto et al., Anal Biochem. 1998 258:53-8; Brenza et al., Proc Natl Acad Sci USA. 1998 95:1387-91; and Takeyama et al., Science. 1997 277:1827-30.

### SUMMARY OF THE INVENTION

The present invention provides peptides which are characterized by having a biological activity that increases 25-hydroxyvitamin D3 1α-hydroxylase activity in a cell, thereby increasing calcitriol (active vitamin D or 1α,25 dihydroxyvitamin D3) levels. The peptides have a sequence related to the contiguous sequence defined by residues 242 to 264 in the naturally occurring matrix extracellular phosphoglycoprotein (PHEX). Methods of modulating 25-hydroxyvitamin 1α-hydroxylase gene expression and calcitriol levels using the subject peptides are also provided. Also provided are kits for practicing the subject methods. The subject compositions and methods find use in a variety of application, including the treatment of vitamin D-related disorders, such as Paget's Disease, rickets, osteoporosis, renal osteodystrophy, and psoriasis.

In many embodiments, a subject peptide comprises the amino acid sequence TDLQERGDNDISPFSGDGQPFKD (SEQ ID NO:1), or a variant of thereof, and may have the sequence of any of the peptides of SEQ ID NOS:2-18. The subject polypeptides may have a sequence that is at least 60% related to at least 15 contiguous amino acid residues of SEQ ID NO:1, or may have the formula:

RX₁X₂NDX₃X₄X₅PFSX₆X₇Q

where X₁-X₇ are independently any amino acid.

The peptide of SEQ ID NO.1-18 and variants thereof significantly induce 1α-hydroxylase gene expression. Since the 1α-hydroxylase gene encodes the enzyme to catalyze the final step in the biosynthetic pathway of calcitriol, any increase in its expression increases the rate of synthesis of calcitriol in the cells which results in an increase in cellular and circulating levels of calcitriol. Increasing the cellular and circulating concentrations of calcitriol is a feature of the subject methods. A feature of the subject peptides is that they do not significantly induce the expression of 24-hydroxylase, an enzyme which increases the flux through the vitamin D pathway, thereby decreasing the amount of available calcitriol in a cell, or in circulation. In addition, the peptides of the present invention operate in a signal transduction pathway independent of the parathyroid hormone signal transduction pathway. The peptides of the present invention, therefore may be used to increase calcitriol levels within a host to treat a host with a vitamin D-related disorder.

Further, the peptides of the invention are useful to timely and selectively upregulate one or more target genes operably linked to the promoter of a 1α-hydroxylase gene.

Formulations containing the peptides in a therapeutically effective amount to treat or cure vitamin D-related conditions are also provided. These formulations are preferably injectable formulations.

Other aspects of the invention are peptides and peptide analogs having at least about 60% identity to the sequence TDLQERGDNDISPFSGDGQPFKD (SEQ ID NO:1).

Yet another aspect of the invention is a method for the treatment of diseases connected with vitamin D metabolism or caused by vitamin D deficiency carried out by administration of a formulation comprising a peptide or peptide analog having at least about 60% to the sequence TDLQERGDNDISPFSGDGQPFKD (SEQ ID NO:1).

Furthermore, subcutaneous injected peptide of SEQ ID NO.1 into the monkeys immediately entered their circulation, retained their biologically active levels to induce 1α-hydroxylase at cellular level, and dose dependently and significantly elevated the serum levels of calctriol.

These and other objects, aspects, features and advantages will become apparent to those skilled in the art upon reading this disclosure.

### BRIEF DESCRIPTION OF THE FIGURES

FIG. 1A and FIG. 1B are bar charts comparing the effects of the peptide of SEQ ID NO.1 at 0 ng/ml, 100 ng/ml or 500 ng/ml on the expression levels of 1α-hydroxylase mRNA in CL-8 cells grown in culture without 100 nmol parathyroid hormone (PTH) in FIG. 1A and with 100 nmol parathyroid hormone (PTH) in FIG. 1B.

FIG. 2 shows a bar chart comparing the effects of H-89, a protein kinase A (PKA) inhibitor, on the induction 1α-hydroxylase mRNA in CL-8 cells grown in culture with 100 nmol PTH, 100 ng/ml the peptide of SEQ ID NO.1 or 500 ng/ml of the peptide of SEQ ID NO.1.

FIG. 3 shows a bar chart comparing the effects of the peptide of SEQ ID NO.1 at 0 ng/ml, 100 ng/ml or 500 ng/ml on the expression levels of 24-hydroxylase mRNA in CL-8 cells grown in culture.

FIG. 4 shows a bar chart comparing the effects of 1,25-dihydroxyvitamin D3 on the expression levels of 24-hydroxylase mRNA in CL-8 cells grown in culture, without the peptide of SEQ ID NO.1, or with the peptide of SEQ ID NO.1 at 100 ng/ml or 500 ng/ml.

FIG. 5 is a multiple sequence alignment of subject peptide sequences.

FIG. 6 is a bar chart comparing the effects of the different doses of the peptide of SEQ ID NO. 1 on the serum levels of 1,25 Dihydroxy Vitamin D3 when it was subcutaneously injected into the monkeys at 0 mg/kg, 1 mg/kg, or 50 mg/kg.

FIG. 7 is a line chart demonstrating the blood levels of the peptide of SEQ ID NO. 1 when it was subcutaneously injected into the monkeys at 50 mg/kg.

### DEFINITIONS

The terms “treat”, “treating”, “treatment” and the like are used interchangeably herein and mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed the disease such as enhancing the effect of vitamin D. “Treating” as used herein covers treating a disease in a vertebrate and particularly a mammal and most particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

The invention is particularly directed towards peptides which make it possible to treat patient's which have experienced bone loss or which would be expected to experience skeletal loss and thus is particularly directed towards preventing, inhibiting, or relieving the effects of skeletal loss. A subject is “treated” provided the subject experiences a therapeutically detectable and beneficial effect which may be measured based on a variety of different criteria including increased skeletal growth, increased skeletal strength or other characteristics generally understood by those skilled in the art to be desirable with respect to the treatment of diseases related to skeletal tissues.

The term “peptidic compound”, as used herein, intends a compound comprising units that are linked to one another primarily, but not exclusively, by peptide bonds. The units typically comprise coded amino acid residues, non-coded amino acid residues, and/or peptidomimetics. The term “peptide” as used herein refers to any compound produced by amide formation between a carboxyl group of one amino acid and an amino group of another. The peptidic compounds may be polymers of: (a) naturally occurring, coded or non-coded, amino acid residues; (b) polymers of non-naturally occurring amino acid residues, e.g. N-substituted glycines, amino acid substitutes, etc.; or (c) polymers of both naturally occurring and non-naturally occurring amino acid residues/substitutes. The term includes synthetic peptides. In other words, the subject peptidic compounds may be peptides or peptoids. Peptoid compounds and methods for their preparation are described in WO 91/19735, the disclosure of which is herein incorporated by reference. Amino acids are sometimes referred to herein by standard three-letter symbols (see, e.g., pages 58-59, “Biochemistry” Second Ed., Voet and Voet, eds. (1995) John Wiley & Sons, Inc.).

The term “effective amount”, “therapeutic amount”, “therapeutically effective amount” and the like are used interchangeably here to describe an amount sufficient to effect a treatment, e.g. a beneficial or desired clinical results. An effective amount can be administered in one or more administrations.

The term “skeletal loss” refers to any situation in which skeletal mass, substance or matrix or any component of the skeleton, such as calcium and phosphate, is decreased or the bone is weakened such as in terms of its ability to resist being broken.

The term “skeleton” includes both bone and teeth. In the same manner, the term “skeletal” means both bone and teeth.

The term “osteoporosis” is intended to refer to any condition involving bone loss, i.e. involving a reduction in the amount of bone mass or substance resulting from any cause. The term particularly results in a bone loss resulting from demineralization of the bone, post menopausal or peri-menopausal estrogen decrease or nerve damage.

The term “subject” refers to any vertebrate, particularly any mammal and most particularly including human subjects.

The term “isolated” refers to material removed from its natural milieu or original environment (e.g., the natural environment if it is naturally occurring), and thus is altered “by the hand of man” from its natural state. For example, an isolated polypeptide could be part of a composition of matter, or could be contained within a cell, and still be “isolated” because that composition of matter, or particular cell is not the original environment of the polypeptide.

The term “vitamin D-related condition” as used herein refers to a condition associated with a deficiency in vitamin D, and also refers to conditions that are treatable by increasing vitamin D. Vitamin D-related disorders include rickets, osteomalacia, osteoporosis, Paget's disease, osteopenia, osteosclerosis or renal osteodystrophy, acute osteosclerosis, psoriasis, medullary carcinoma, Alzheimer's disease hyperparathyroidism, hypoparathyroidism, pseudoparathyroidism, secondary parathyroidism, diabetes, cirrhosis, obstructive jaundice or drug-induced metabolism, glucocorticoid antagonism, hypercalcemia, malabsorption syndrome, steatorrhea, chronic renal disease, hypophosphatemic vitamin D-resistant rickets, vitamin D-dependent rickets, rickets type I, rickets type II sarcoidosis, leukemia, prostate cancer, breast cancer, colon cancer, organ transplantation or an immunodisorder. Vitamin D-related conditions also include bone fracture, and various dental disorders.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

### DETAILED DESCRIPTION OF SPECIFIC EMBODIMENTS

The present invention provides peptides which are characterized by having a biological activity that increases 25-hydroxyvitamin D3 1α-hydroxylase activity in a cell, thereby increasing calcitriol (active vitamin D) levels. The peptides have a sequence related to the contiguous sequence defined by residues 242 to 264 in the naturally occurring matrix extracellular phosphoglycoprotein (PHEX). Methods of modulating 25-hydroxyvitamin 1α-hydroxylase gene expression and calcitriol levels using the subject peptides are also provided. Also provided are kits for practicing the subject methods. The subject compositions and methods find use in a variety of application, including the treatment of vitamin D-related disorders, such as Paget's Disease, rickets, osteoporosis, renal osteodystrophy, and psoriasis.

Before the peptides, analogs, formulations, and methodology of the present invention are described, it is to be understood that this invention is not limited to any particular embodiment described, as such may, of course, vary. It is also to be understood that the terminology used herein is with the purpose of describing particular embodiments only, and is not intended to limit the scope of the present invention that will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a peptide” includes a plurality of such peptides and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Peptide Compositions

The subject invention provides peptides that are characterized by a biological activity that modulates (i.e. increases or decreases) expression of 25-hydroxyvitamin D3 1α-hydroxylase, an enzyme that catalyzes the final step of active vitamin D biosynthesis. Such polypeptides are termed “dentonin” polypeptides and are generally related to the contiguous sequence of amino acids defined by residues 242 to 264 in the naturally occurring matrix extracellular phosphoglycoprotein, as defined by NCBI Accession number AAK70343. As such, a dentonin peptide comprises the sequence TDLQERGDNDISPFSGDGQPFKD (SEQ ID NO:1), or is a variant peptide related to SEQ ID NO:1 that retains the ability to modulate 1α-hydroxylase activity. The ability to modulate 1α-hydroxylase activity is “dentonin activity”, an increase in which usually does not significantly increase 24-hydroxylase activity.

Dentonin peptides that are variants of SEQ ID NO:1 include TDLQERDGNDISPFSGDGQPFKD (SEQ ID NO.2), TDLQERGDNDISPFSDGDQPFKD (SEQ ID NO.3), TDLQERGDNDMSPFSGDGQPFKD (SEQ ID NO.4), and PDLLVRGDNDVPPFSGDGQHFMH (SEQ ID NO.5), and the smaller fragments of these peptides, e.g., TDLQERGDNDISPFSGDGQPF (SEQ ID NO.6), TDLQERGDNDISPFSGDGQ (SEQ ID NO.7), DLQERGDNDISPFSGDGQPF (SEQ ID NO.8), ERGDNDISPFSGDGQPFKD (SEQ ID NO.9), ERGDNDISPFSGDGQ (SEQ ID NO.10), DLQERDGNDISPFSGDGQPFKD (SEQ ID NO.11), TDLQERDGNDISPFSGDGQPF (SEQ ID NO.12), DLQERDGNDISPFSGDGQPF (SEQ ID NO.13), ERDGNDISPFSGDGQ (SEQ ID NO.14), ERGDNDMSPFSGDGQ (SEQ ID NO.15), VRGDNDVPPFSGDGQ (SEQ ID NO.16), CTDLQERGDNDISPFSGDGQPFKD (SEQ ID NO:17) and TDLQERGDNDISPFSGDGQPFKDC (SEQ ID NO:18).

In general, dentonin peptides have at least 50% sequence identity, at least 55% sequence identity, at least 60% sequence identity at least 65% sequence identity at least 70% sequence identity at least 75% sequence identity at least 80% sequence identity at least 85% sequence identity at least 90% sequence identity at least 95% sequence identity or 100% sequence identity with any one of SEQ ID NOS:1-18, or a fragment of any one of SEQ ID NOS:1-18. A fragment of any one of SEQ ID NOS:1-18 may be 6, 7, 8, 9, 10, 11, 12, 13, 14, 15, 16, 17, 18, 19, 20, 21, or 22 contiguous amino acids in length.

A dentonin peptide may about 10 amino acids, about 12 amino acids, about 14 amino acids, about 16 amino acids, about 18 amino acids, about 20 amino acids, about 23 amino acids, about 25 amino acids, about 30 amino acids, about 35 amino acids, about 40 amino acids, about 45 amino acids, about 50 amino acids, and may, of course, be fused or otherwise covalently bonded to other peptides to form a fusion protein. As such, a dentonin polypeptide may longer than 50 amino acids in length, as long as it retains its dentonin activity.

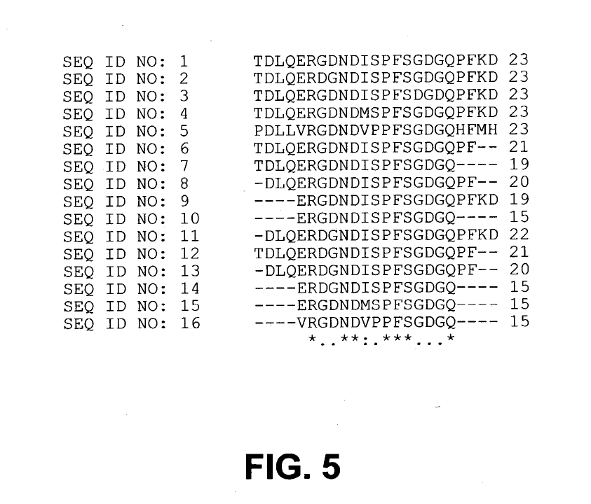

Active dentonin peptide sequences were subjected to CLUSTAL analysis, and the resulting sequence alignment and consensus formula are displayed in FIG. 5).

In many embodiments, dentonin peptides are described by the formula (shown in FIG. 5 using the standard single amino acid code):

ZₙRX₁X₂NDX₃X₄X₅PFSX₆X₇QZₘ,

where X₁-X₇ are any amino acid. In certain embodiments, dentonin peptides may be described by the formula:

ZₙRX₁X₂NDX₃X₄X₅PFSX₆X₇QZₘ,

where X₁-X₂ and X₄-X₇ are any amino acid and X₃ is a M, L, I or V. In certain other embodiments, the formula for dentonin peptides is:

ZₙRX₁X₂NDX₃X₄X₅PFSX₆X₇QZₘ,

where X₁ is D or G, X₂ is D or G, X₃ is I, V or M, X₄ is P or S, X₅ is G or D, X₆ is D or G, and X₇ is D or G. In each of the above formula, the letters correspond to amino acids using the standard single letter amino acid code, and where Zₘ and Zₙ are each a contiguous series of any amino acids, where n or m each may independently be 0 (i.e. no amino acid), 1, 2, 3, 4, 5, 6, 7, 8, 9, 10, more than about 10, more than about 20, more than about 30, more than about 40, more than about 50, more than about 80, or more than about 100 or more, up to about 500 or more, amino acids.

In addition to the specific dentonin described above, further variants may be found from species other than humans by examining the amino acids sequences matrix extracellular phosphoglycoproteins of, e.g., rat (NCBI Accession number NP_{—}077056), mouse (NCBI Accession numbers NP_{—}444402 and AAK70342), and macaque (NCBI Accession number BAB01638) for dentonin fragment. Further dentonin variants may be generated by substituting amino acids at equivalent positions (e.g., as determined by a sequence alignment e.g., the sequence alignment of FIG. 5), between different known variants (e.g., the variants shown in FIG. 5), or the variants described in the rat, mouse and macaque matrix extracellular phosphoglycoproteins.

The dentonin proteins of the subject invention are usually present in a non-naturally occurring environment, i.e., are separated from their naturally occurring environment. In certain embodiments, the subject dentonin is present in a composition that is purified for dentonin as compared to dentonin in its naturally occurring environment. As such, purified dentonin is provided, where by purified is meant that dentonin is present in a composition that is substantially free of non-dentonin proteins, where by substantially free is meant that less than 90%, usually less than 60% and more usually less than 50% of the composition is made up of non-dentonin proteins. For compositions that are enriched for dentonin proteins, such compositions will exhibit dentonin activity.

In certain embodiments of interest, the dentonin protein is present in a composition that is substantially free of the constituents that are present in its naturally occurring environment. For example, a dentonin-comprising composition according to the subject invention in this embodiment will be substantially, if not completely, free of those other biological constituents, such as proteins, carbohydrates, lipids, etc., with which it is present in its natural environment. As such, protein compositions of these embodiments will necessarily differ from those that are prepared by purifying the protein from a naturally occurring source, where at least trace amounts of the proteins constituents will still be present in the composition prepared from the naturally occurring source.

The dentonin of the subject invention may also be present as an isolate, by which is meant that the dentonin is substantially free of both non-dentonin proteins and other naturally occurring biologic molecules, such as oligosaccharides, polynucleotides and fragments thereof, and the like, where substantially free in this instance means that less than 70%, usually less than 60% and more usually less than 50% of the composition containing the isolated dentonin is a non-dentonin naturally occurring biological molecule. In certain embodiments, the dentonin is present in substantially pure form, where by substantially pure form is meant at least 95%, usually at least 97% and more usually at least 99% pure.

In one embodiment of the invention, dentonin consists essentially of a polypeptide sequence set forth in any one of SEQ ID NO:1-18, or a variant thereof. By “consisting essentially of” in the context of a polypeptide described herein, it is meant that the polypeptide is composed of the sequence set forth in the sequence listing, which sequence may be flanked by one or more amino acid or other residues that do not materially affect the basic characteristic(s) of the polypeptide.

Dentonin Analogs, Derivatives and Mimetics

One skilled in the art may prepare peptides with dentonin activity by modifying sequence of a peptide described above by making single or multiple amino acid substitutions, additions, or deletions. These changes are usually of a minor nature, such as conservative amino acid substitutions, that do not significantly affect the folding or activity of the peptide. For instance, one polar amino acid, such as threonine, may be substituted for another polar amino acid, such as serine; or one acidic amino acid, such as aspartic acid, may be substituted for another acidic amino acid, such as glutamic acid; or a basic amino acid, such as lysine, arginine, or histidien, may be substituted for another basic amino acid; or a non-polar amino acid, such as alanine, leucine or isoleucine, may be substituted for another non-polar amino acid. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990). Of course, the number of amino acid substitutions a skilled artisan would make depends on many factors. Moreover, amino acids in dentonin that are essential for function can be identified by methods known in the art, such as site-directed mutagenesis or alanine-scanning mutagenesis. (Cunningham & Wells, Science 244:1081-1085 (1989)). The latter procedure introduces single alanine mutations at every residue in the molecule. The resultant mutant molecules are then tested for biological activity.

The invention additionally, encompasses polypeptides which are differentially modified e.g. during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are chemically modified derivatives of the polypeptides of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The 5 polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200; 500; 1000; 1500; 2000; .2500; 3000; 3500; 4000; 4500; 5000; 5500; 6000; 6500; 7000; 7500; 8000; 8500; 9000; 9500; 10,000; 10,500; 11,000; 11,500; 12,000; 12,500; 13,000; 13,500; 14,000; 14,500; 15,000; 15,500; 16,000; 16,500; 17,000; 17,500; 18,000; 18,500; 19,000; 19,500; 20,000; 25,000; 30,000; 35,000; 40,000; 50,000; 55,000; 60,000; 65,000; 70,000; 75,000; 80,000; 85,000; 90,000; 95,000; or 100,000 kDa.

Pegylation of the proteins of the invention may be accomplished by any number of means. For example, polyethylene glycol may be attached to the protein either directly or by an intervening linker. Linkerless systems for attaching polyethylene glycol to proteins are described in Delgado et ai, Crit. Rev. Thera. Drug Carrier Sys. 9:249-304 (1992); Francis et al.. Intern. J. of Hematol. 65:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of 5 proteins without an intervening linker employs tresylated MPEG, which is produced by the modification of monmethoxy polyethylene glycol (MPEG) using tresylchloride (ClSO₂CH₂CFl). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Further contemplated are target modifications of amino acids of the subject polypeptides with an organic derivatizing agent capable of reacting with selected side chains or the N- or C-terminal residues of a subject polypeptide. Such agents include, e.g. 1,1-bis(diazoacetyl)-2-penylethane, glutaraldehyde, N-hydroxysaccinibide esters, and the like.

Other modifications may be found in PCT publication WO00/55173.

Nucleic Acids, Vectors and Host Cells

The invention further provides isolated nucleic acids comprising a nucleotide sequence encoding dentonin, and vectors and host cells containing the same. In many embodiments, a subject nucleic acid comprises a coding sequence for dentonin. Since the genetic code is known, and the sequence of a dentonin is described herein, the design and production of these nucleic acids is well within the skill of an artisan (see, e.g., Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.).

The invention further provides vectors (also referred to as “constructs”) comprising a subject nucleic acid. In many embodiments of the invention, nucleic acid sequences encoding dentonin will be expressed in a host after the sequences have been operably linked to an expression control sequence, including, e.g., a promoter. The subject nucleic acids are also typically placed in an expression vector that can replicable in a host organisms either as an episome or as an integral part of the host chromosomal DNA. Commonly, expression vectors will contain selection markers, e.g., tetracycline, neomycin, etc., to permit detection of those cells transformed with the desired DNA sequences (see, e.g., U.S. Pat. No. 4,704,362, which is incorporated herein by reference). Vectors, including single and dual expression cassette vectors, are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes (human artificial chromosomes, bacterial artificial chromosomes, yeast artificial chromosomes, etc.), mini-chromosomes, and the like.

The expression vector will usually provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

The invention further provides host cells, including isolated in vitro host cells and in vivo host cells, that comprise a nucleic acid or a vector of the invention. Suitable host cells, including bacterial, yeast, insect and mammalian host cells may be found in PCT publication WO 00/55173.

Method of Production

Dentonin can be isolated or synthesized using methods well known in the art. Such methods include recombinant DNA methods and chemical synthesis methods for production of a peptide. Recombinant methods of producing a peptide through expression of a nucleic acid sequence encoding the peptide in a suitable host cell are well known in the art and are described, for example, in Sambrook et al. (Molecular Cloning: A Laboratory Manual, 2nd Ed, Vols 1 to 3, Cold Spring Harbor Laboratory Press, New York (1989)), which is incorporated herein by reference.

Specific expression systems of interest include bacterial, yeast, insect cell and mammalian cell derived expression systems. Representative systems from each of these categories are provided below:

Recombinant Methods

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. Pat. No. RE 30,985.

Chemical Synthesis

Dentonin can be produced by chemical synthesis, for example, by the solid phase peptide synthesis method of Merrifield et al. (J. Am. Chem. Soc. 85:2149 (1964)), which is incorporated herein by reference. Standard solution methods well known in the art also can be used to synthesize a peptide useful in the invention (see, for example, Bodanszky, Principles of Peptide Synthesis, Springer-Verlag, Berlin (1984) and Bodanszky, Peptide Chemistry, Springer-Verlag, Berlin (1993), each of which is incorporated herein by reference). A newly synthesized peptide can be purified, for example, by high performance liquid chromatography (HPLC), and can be characterized using, for example, mass spectrometry or amino acid sequence analysis.

For example, polypeptides of the invention can be chemically synthesized the methods of, e.g., see Creighton (1983, Proteins: Structures and Molecular Principles, W.H. Freeman & Co., N.Y.) or Hunkapiller et al. (Nature, 310:105-111 (1984)). In many embodiments, a polypeptide corresponding to a fragment of a polypeptide can be synthesized by use of a peptide synthesizer (e.g. a peptide synthesizer of Applied Biosystems, Foster City, Calif.). Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, 5 hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acids can be D (dextrorotary) or L (levorotary). Further methods and amino acid analogs for peptide synthesis may be found in Chan et al, (Fmoc solid phase peptide synthesis: A Practical Approach, Oxford University Press, 2000) and Bodanszky (Principles of Peptide Synthesis, Springer Verlag, 2nd edition, 1993).

Methods of Modulating Calcitriol

The subject invention provides methods of increasing 25-hydroxyvitamin D3 1α-hydroxylase (“1α-hydroxylase”) activity and/or calicitriol levels in a cell. In many embodiments, the methods include contacting a cell an effective amount of one or more active agents that modulate 1α-hydroxylase activity in the cell to modulate calcitriol amounts in the cell or host. In certain embodiments, the one or more active agents include dentonin. In some embodiments the desired increase is an increase in 1α-hydroxylase mRNA levels, 1α-hydroxylase protein levels, or 1α-hydroxylase enzyme activity.

By “1α-hydroxylase activity” is meant the activity of a 1α-hydroxylase protein, where representative 1α-hydroxylase proteins are disclosed in GenBank Accession Nos. NP_{—}000776, NP_{—}446215, or O35084, and discussed in Fu et al. (DNA Cell Biol. 16:1499-507, 1997) and Kitanaka et al. (N. Engl J Med. 338:653-61, 1998). Assays for determining the activity of 1α-hydroxylase include those of Nakamura FEBS Lett. 419:45-8, 1997) and Eto (Anal Biochem. 258:53-8, 1998), and may also include methods for determining 1α-hydroxylase gene expression, e.g. RT-PCR, RNA or protein gel blotting, RNA hybridization, etc., as is known in the art (e.g., Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) and further described below.

In certain embodiments 1α-hydroxylase activity and/or calcitriol amounts are increased by at least about 10%, at least about 30%, at least about 50%, at least about 70%, at least about 90%, at least about 100%, at least about 150%, at least about 200%, at least about 250%, at least about 300%, at least about 400%, at least about 500%, at least about 700%, at least about 1000%, at least about 2000%, at least about 5000% or more, as compared to a baseline 1α-hydroxylase activity level, e.g., that observed in the cell or host prior to contact with dentonin.

In many embodiments, 1α-hydroxylase activity and/or calcitriol amounts are increased in any site in which suitable calcitriol precursor molecules are available, for example, kidney cells.

In certain embodiments, an active agent increases 1α-hydroxylase activity to a greater extent than is increases the activity of vitamin D 24-hydroxylase (“25 hydroxylase”, e.g. the activity of the protein described by NCBI accession numbers NP_{—}000773, AAB03776, CAB91829, or CAB91829). As such, in most embodiments, if an active agent is contacted with a cell, it will increase the levels of 1α-hydroxylase activity (e.g. the level of 1α-hydroxylase mRNA) by a certain amount, and will increase the activity of 25 hydroxylase (e.g. the level of 25 hydroxylase mRNA) by an undetectable level, by less than about 1%, by less than about 5%, by less than about 10%, by less than about 20%, by less than about 30%, by less than about 40%, or by less than about 50% of that amount, as compared to baseline activity levels in a cell or host. For example, if 1α-hydroxylase mRNA levels are increased by 500%, the levels of 25 hydroxylase mRNA may be induced by 5% of that amount (25%). In certain embodiments there is no detectable increase in 25 hydroxylase activity when a cell or is contacted with an active agent (e.g., dentonin).

In many embodiments, the methods include administering to a host an effective amount of one or more active agents that modulate 1α-hydroxylase activity in the host to calcitriol levels in the host. The active agent may be a variety of different compounds, including: polynucleotide compositions (e.g., coding sequences for dentonin, etc.) polypeptide compositions (e.g., dentonin polypeptide, etc.), and, naturally occurring or synthetic small molecule compounds, etc.

In certain embodiments, the active agents administered to the host are polynucleotide or nucleic acid compositions. The nucleic acids may be coding sequences, e.g., genes, gene fragments etc. for dentonni, which may be present in expression vectors, where such vectors generally have convenient restriction sites located near the promoter sequence to provide for the insertion of nucleic acid sequences. Transcription cassettes may be prepared that include a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells to express dentonin, usually for a period of at least about one day, more usually for a period of at least about several days to several weeks.

Formulations

In practicing the subject methods, an effective amount of the active agent is administered to the host, where the term “effective amount” means a dosage sufficient to produce a desired result, where the desired result is the desired modulation, e.g., enhancement, reduction, of 1α-hydroxylase activity and/or calcitriol levels.

In practicing the subject methods, the active agent or agents are typically administered to the host in a physiologically acceptable delivery vehicle, e.g., as a pharmaceutical preparation. A variety of representative formulations, dosages, routes of administration for candidate agents, nucleic acid delivery vehicles and nucleic acid formulations for nucleic acid delivery are described below.

Formulations, Dosages, and Routes of Administration

The invention provides formulations, including pharmaceutical formulations, that include an agent which modulates 1α-hydroxylase activity and/or calcitriol levels in a host. In general, a formulation comprises an effective amount of an agent that modulates 1α-hydroxylase activity activity in a host. An “effective amount” refers to an amount that is sufficient to produce a desired result, e.g., increase in a level of 1α-hydroxylase activity expression and/or increase in calcitriol, etc. In many embodiments, the desired result is at least a reduction or increase in a phenotype as compared to a control such that the phenotype is more similar to normal.

Formulations

In the subject methods, the active agent(s) may be administered to the host using any convenient means capable of resulting in the desired increase in 1α-hydroxylase activity and/or calcitriol levels in a host.

Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of an agent that reduces a symptom of a vitamin D-related disorder in a subject animal.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of Administration

Conventional and pharmaceutically acceptable routes of administration include intranasal, intramuscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses. In certain embodiments, formulations are administered to a kidney of a host, through, e.g., direct injection, or injection into a renal artery.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrastemal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as an vitamin D-related disorder and psychological trauma associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A subject polynucleotide can be delivered as a naked polynucleotide, or associated with (complexed with) a delivery vehicle. “Associated with”, or “complexed with”, encompasses both covalent and non-covalent interaction of a polynucleotide with a given delivery vehicle.

Nucleic Acid Delivery Vehicles

In certain embodiment, an agent is a nucleic acid. Nucleic acids may be delivered using several different vehicles, including viral and non-viral delivery vehicles.

Viral Delivery Vehicles

A subject polynucleotide can be associated with viral delivery vehicles. As used herein, a “viral delivery vehicle” intends that the polynucleotide to be delivered is encapsidated in a viral particle.

Numerous viral genomes useful in in vivo transformation and gene therapy are known in the art, or can be readily constructed given the skill and knowledge in the art. Included are replication competent, replication deficient, and replication conditional viruses. Viral vectors include adenovirus, mumps virus, a retrovirus, adeno-associated virus, herpes simplex virus (HSV), cytomegalovirus (CMV), vaccinia virus, and poliovirus, and non-replicative mutants/variants of the foregoing. In some embodiments, a replication-deficient virus is capable of infecting slowly replicating and/or terminally differentiated cells, since the respiratory tract is primarily composed of these cell types. For example, adenovirus efficiently infects slowly replicating and/or terminally differentiated cells. In some embodiments, the viral genome itself, or a protein on the viral surface, is specific or substantially specific for cells of the targeted cell. A viral genome can be designed to be target cell-specific by inclusion of cell type-specific promoters and/or enhancers operably linked to a gene(s) essential for viral replication.

Where a replication-deficient virus is used as the viral genome, the production of virus particles containing either DNA or RNA corresponding to the polynucleotide of interest can be produced by introducing the viral construct into a recombinant cell line which provides the missing components essential for viral replication and/or production. Preferably, transformation of the recombinant cell line with the recombinant viral genome will not result in production of replication-competent viruses, e.g., by homologous recombination of the viral sequences of the recombinant cell line into the introduced viral genome. Methods for production of replication-deficient viral particles containing a nucleic acid of interest are well known in the art and are described in, for example, Rosenfeld et al., Science 252:431-434, 1991 and Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus). Methods and materials for manipulation of the mumps virus genome, characterization of mumps virus genes responsible for viral fusion and viral replication, and the structure and sequence of the mumps viral genome are described in Tanabayashi et al., J. Virol. 67:2928-2931, 1993; Takeuchi et al., Archiv. Virol., 128:177-183, 1993; Tanabayashi et al., Virol. 187:801-804, 1992; Kawano et al., Virol., 179:857-861, 1990; Elango et al., J. Gen. Virol. 69:2893-28900, 1988.

Non-Viral Delivery Vehicles

A subject polynucleotide can be administered using a non-viral delivery vehicle. “Non-viral delivery vehicle” (also referred to herein as “non-viral vector”) as used herein is meant to include chemical formulations containing naked or condensed polynucleotides (e.g, a formulation of polynucleotides and cationic compounds (e.g., dextran sulfate)), and naked or condensed polynucleotides mixed with an adjuvant such as a viral particle (i.e., the polynucleotide of interest is not contained within the viral particle, but the transforming formulation is composed of both naked polynucleotides and viral particles (e.g., adenovirus particles) (see, e.g., Curiel et al. 1992 Am. J. Respir. Cell Mol. Biol. 6:247-52)). Thus “non-viral delivery vehicle” can include vectors composed of polynucleotides plus viral particles where the viral particles do not contain the polynucleotide of interest. “Non-viral delivery vehicles” include bacterial plasmids, viral genomes or portions thereof, wherein the polynucleotide to be delivered is not encapsidated or contained within a viral particle, and constructs comprising portions of viral genomes and portions of bacterial plasmids and/or bacteriophages. The term also encompasses natural and synthetic polymers and co-polymers. The term further encompasses lipid-based vehicles. Lipid-based vehicles include cationic liposomes such as disclosed by Felgner et al (U.S. Pat. Nos. 5,264,618 and 5,459,127; PNAS 84:7413-7417, 1987; Annals N.Y. Acad Sci. 772:126-139, 1995); they may also consist of neutral or negatively charged phospholipids or mixtures thereof including artificial viral envelopes as disclosed by Schreier et al. (U.S. Pat. Nos. 5,252,348 and 5,766,625).

Non-viral delivery vehicles include polymer-based carriers. Polymer-based carriers may include natural and synthetic polymers and co-polymers. Preferably, the polymers are biodegradable, or can be readily eliminated from the subject. Naturally occurring polymers include polypeptides and polysaccharides. Synthetic polymers include, but are not limited to, polylysines, and polyethyleneimines (PEI; Boussif et al., PNAS 92:7297-7301, 1995) which molecules can also serve as condensing agents. These carriers may be dissolved, dispersed or suspended in a dispersion liquid such as water, ethanol, saline solutions and mixtures thereof. A wide variety of synthetic polymers are known in the art and can be used.

“Non-viral delivery vehicles” further include bacteria. The use of various bacteria as delivery vehicles for polynucleotides has been described. Any known bacterium can be used as a delivery vehicle, including, but not limited to non-pathogenic strains of Staphylococcus, Salmonella, and the like.

Formulations for Nucleic Acid Delivery

The polynucleotide to be delivered can be formulated as a DNA- or RNA-liposome complex formulation. Such complexes comprise a mixture of lipids which bind to genetic material (DNA or RNA) by means of cationic charge (electrostatic interaction). Cationic liposomes which may be used in the present invention include 3β-[N-(N′, N′-dimethyl-aminoethane)-carbamoyl]-cholesterol (DC-Chol), 1,2-bis(oleoyloxy-3-trimethylammonio-propane (DOTAP) (see, for example, WO 98/07408), lysinylphosphatidylethanolamine (L-PE), lipopolyamines such as lipospermine, N-(2-hydroxyethyl)-N,N-dimethyl-2,3-bis(dodecyloxy)-1-propanaminium bromide, dimethyl dioctadecyl ammonium bromide (DDAB), dioleoylphosphatidyl ethanolamine (DOPE), dioleoylphosphatidyl choline (DOPC), N(1,2,3-dioleyloxy) propyl-N,N,N-triethylammonium (DOTMA), DOSPA, DMRIE, GL-67, GL-89, Lipofectin, and Lipofectamine (Thiery et al. (1997) Gene Ther. 4:226-237; Felgner et al., Annals N.Y. Acad. Sci. 772:126-139, 1995; Eastman et al., Hum. Gene Ther. 8:765-773, 1997). Polynucleotide/lipid formulations described in U.S. Pat. No. 5,858,784 can also be used in the methods described herein. Many of these lipids are commercially available from, for example, Boehringer-Mannheim, and Avanti Polar Lipids (Birmingham, Ala.). Also encompassed are the cationic phospholipids found in U.S. Pat. Nos. 5,264,618, 5,223,263 and 5,459,127. Other suitable phospholipids which may be used include phosphatidylcholine, phosphatidylserine, phosphatidylethanolamine, sphingomyelin, phosphatidylinositol, and the like. Cholesterol may also be included.

Methods of Modulating Gene Expression in a Host Cell

The invention further provides methods of modulating nucleic acid expression in a host cell. In general, these methods involve modulating the expression of one or more target nucleic acids operably linked to a 1α-hydroxylase promoter, (examples of which promoter is described in Shinki et al. PNAS 1999 96: 6988-6993; Yoshida et al., J. Am Soc Nephrol. 2002; Brenza et al., Proc Natl Acad Sci USA. 1998 95:1387-91 and in SEQ ID NO:19) in a cell. In certain embodiments, a vector construct comprising a target nucleic acid operably linked to a 1α-hydroxylase promoter is prepared to form an expression cassette, and the vector is transferred into a cell, particularly a kidney cell. The cell is then contacted with dentonin to effect expression of the nucleic acid. In certain embodiments, the nucleic acid encodes a polypeptide, and, as such, such methods are useful in the production of one or polypeptides in a host cell. Methods for making vectors containing expression cassettes and expression systems for such vectors are generally well known in the art (see, e.g., Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.).

In most embodiments, nucleic acids operably linked to a 1α-hydroxylase promoter in a cell will be induced upon contact of the cell with dentonin. In many embodiments, the production of the nucleic acid will increase by at least about 20%, at least about 20%, at least about 40%, at least about 80%, at least about 100%, at least about 150%, at least about 200%, at least about 300%, at least about 400%, at least about 500%, at least about 1000%, to at least about 10,000%, as compared the same expression cassette in a cell in the absence of dentonin. In certain embodiments, the cell is comprised within an animal host, and the methods may be used to induce expression of a nucleic acid in the host.

Such methods may be used to produce a polypeptide in a cell. As such, in some embodiments the invention provides a method of gene therapy. In general, these embodiments involve operably linking a target gene sequence to a promoter of a 1α-hydroxylase gene to form a cassette, transferring the cassette into a mammalian subject and administering to the subject a dentonin formulation, wherein the formulation induce a therapeutically effective expression of the target gene. In certain embodiments, the cassette is transferred to kidney cells of a mammalian subject.

Utility

The subject compositions and methods of increasing 1α-hydroxylase activity and/or calcitriol levels in a host find use in a variety of therapeutic protocols. In general, these protocols involve administering to a host in need of such treatment (e.g. a host suffering from a vitamin D-related condition or a host with a condition treatable by an increase in vitamin D levels) an effective amount of one or more active agents that modulate 1α-hydroxylase activity in the host to increase calcitriol amounts in the host and treat the host.

In some embodiments, the subject compositions and methods may be used to increase calcitriol levels in a host. In general, these embodiments involve administering a dentonin peptide to a host, where the host increases its levels of calcitriol, in particular serum calcitriol, by at least about 20%, at least about 20%, at least about 40%, at least about 80%, at least about 100%, at least about 150%, at least about 200%, at least about 300%, at least about 400%, at least about 500%, at least about 1000%, as compared to a host not administered the dentonin.

By treatment is meant at least an amelioration of a symptom associated with the pathological condition afflicting the host, where amelioration is used in a broad sense to refer to at least a reduction in the magnitude of a parameter, e.g. symptom, associated with the pathological condition being treated, such slow bone healing, weak bones, etc. As such, treatment also includes outcomes where the pathological condition, or at least symptoms associated therewith, are completely inhibited, e.g. prevented from happening, or stopped, e.g. terminated, such that the host no longer suffers from the pathological condition, or at least the symptoms that characterize the pathological condition. For example, where the disease condition is marked by brittle bones, treatment includes at least a reduction in the observed brittleness of the bones, including a restoration of normal bone strength.

A variety of hosts are treatable according to the subject methods. Generally such hosts are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Of particular interest is treatment and prevention of disorders associated with undesirable calcitriol levels, including reduced calcitriol levels caused by, e.g., reduced calcitriol synthesis, increases calcitriol flux, etc. Examples of such disorders include, but are not limited to, rickets, osteomalacia, osteoporosis, Paget's disease, osteopenia, osteosclerosis or renal osteodystrophy, acute osteosclerosis, psoriasis, medullary carcinoma, Alzheimer's disease hyperparathyroidism, hypoparathyroidism, pseudoparathyroidism, secondary parathyroidism, diabetes, cirrhosis, obstructive jaundice or drug-induced metabolism, glucocorticoid antagonism, hypercalcemia, malabsorption syndrome, steatorrhea, chronic renal disease, hypophosphatemic vitamin D-resistant rickets, vitamin D-dependent rickets, rickets type I, rickets type II sarcoidosis, leukemia, prostate cancer, breast cancer, colon cancer, organ transplantation or an immunodisorder. Vitamin D-related conditions also relate to bone fracture healing, and various dental disorders. The subject methods also find use in methods for increasing calcitriol levels in hosts not suffering from a particular vitamin-D related disorder but in which the modulation of calcitriol levels is desired. For example, subject methods may be performed on a host with a desire to avoid contracting such a vitamin-D-related disorder. Subjects of particular interest include those that are prone to vitamin D-related conditions, such as such as women of the age groups of 50-70 years, and women of the age group of 70-90 years, that are, for example, suffering from type 1 or type 2 osteoporosis, respectively.

Kits

Also provided by the subject invention are kits for practicing the subject methods, as described above. The subject kits at least include one or more of a pharmaceutical preparation comprising at least dentonin, as described above. Other optional components of the kit include: a syringe or another administration device. The various components of the kit may be present in separate containers or certain compatible components may be precombined into a single container, as desired. In many embodiments, kits with unit doses of the active agent, e.g. in oral or injectable doses, are provided. In many embodiments the subject composition is contained within a media, such phosphate buffered saline, tris, glycerol, and the like.

In addition to above-mentioned components, the subject kits typically further include instructions for using the components of the kit to practice the subject methods of treating a host in need of such treatment by administering to said host an effective amount of one or more active agents that increase 1α-hydroxylase activity in the host to modulate calcitriol levels in the host and treat the host. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXPERIMENTAL

The following examples are put forth so as to provide those of ordinary skill in the art with a complete disclosure and description of how to make and use the present invention, and are not intended to limit the scope of what the inventors regard as their invention, nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Celsius, and pressure is at or near atmospheric.

Materials and Methods

Cell culture, RNA preparation and cDNA synthesis: CL-8 cells (1.6×10⁶ cells) were placed in 60 mm culture dish, and cultured for 48 hours in 5% FCS D-MEM/HamF-12(1:1) medium. This medium was exchanged to FCS free, 0.5 mM Ca defined medium [D-MEM/HamF-12 (13:87), Insulin 5 μg/ml, Transferrin 5 μg/ml, Na₂SeO₃ 5 ng/ml, T₃ 0.37 nM, EGF 2.5 ng/ml, Hydrocortisone 1 nM]. After 24 hours, the peptide of SEQ ID NO.1 (100 ng/ml or 500 ng/ml) or PTH 100 nM were added and cultured for 5 hours. After treatment, the total cellular contents of each well were used for RNA extraction and reverse transcription (RT). RNA was isolated using ISOGEN (Nippon Gene, Japan) according to the manufacturer's protocol. The purified RNA was redissolved in 10 μl RNase-free water and used for cDNA synthesis by RT. The RT was performed in a 20 μl reaction mixture containing 10 mM Tris buffer, pH8.3, 50 mM KCl, 5 mM MgCl₂, 1 mM each of dATP, dGTP, dCTP and dTTP, 20 U of RNase inhibitor, 1 μM Reverse primer and 5 U of AMV reverse transcriptase. The reaction mixture was incubated at 30° C. for 10 min, 55° C. for 15 min, 95° C. for 5 min followed by 5° C. for 5 min.

Real-time quantitative PCR: mRNA was quantitated for human 1α-OHase gene and β-actin. Theses gene were amplified using primers of oligo synthesis DNA (Takara, Japan). Human 1α-OHase gene primers amplify a 115 bp product [located between bases 1197-1311 of homo sapiens cytochrome P450, subfamily XXVIIB (25-hydroxylvitamin D-1-alpha-hydroxylase), polypeptide 1 (CYP27B1), mitochondrial protein encoded by nuclear gene, mRNA sequence, accession number: NM000785]. β-actin primers amplify a 77 bp product [located between bases 250-326 of mouse muscle melanoma X-actin (Actx), mRNA sequence, accession number: NM007393]. Quantitation of mRNA was performed using Applied Biosystems GeneAmp 5700 sequence detection system with SYBR Green methods.

The primers sets used were: Human 1α-OHase forward: 5′-

| | | |
|---|---|---|
| Human 1α-OHase forward: | 5′-ACTGTACCCTGTGGTACCTG-3′, | (SEQ ID NO:20) |
| Human 1α-OHase reverse: | 5′-CCTTGAAGTGGCATAGTGAC-3′, | (SEQ ID NO:21) |
| β-actin forward: | 5′-AGGCCCAGAGCAAGAGAGGTAT-3′, | (SEQ ID NO:22) |
| β-actin forward: | 5′-CATGTCGTCCCAGTTGGTAACA-3′. | (SEQ ID NO:23) |

### EXAMPLE 1

### Dentonin Peptide Regulates 1α-Hydroxylase mRNA Expression

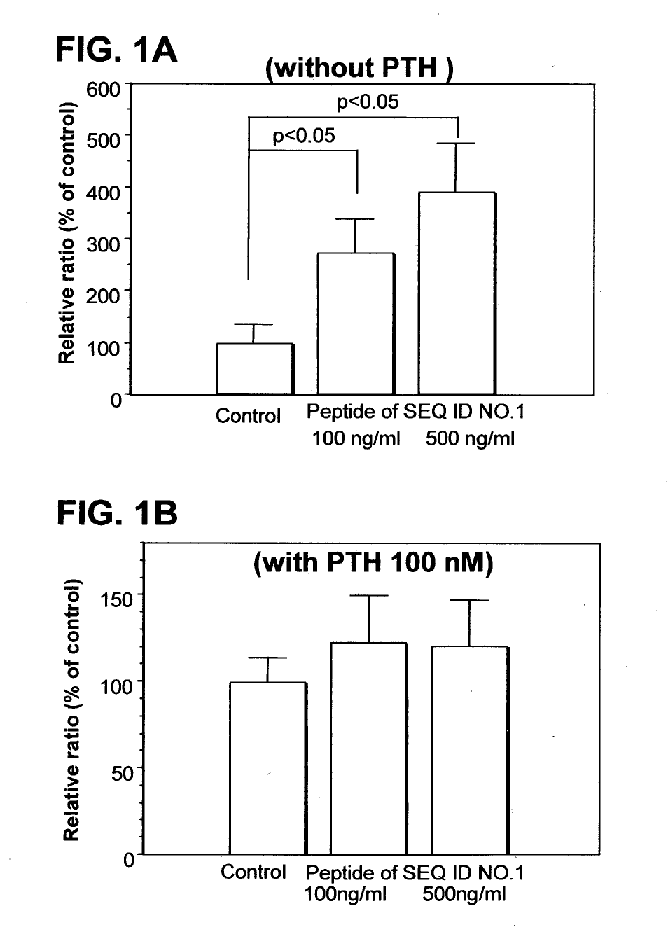

In order to study the effect of the peptide of SEQ ID No. 1 on the regulation of 1α-hydoxylase mRNA expression, CL-8 cells were grown with and without the peptide of SEQ ID No. 1 and the effects of the peptide on 1α-hydroxylase were quantified. FIGS. 1a and 1b shows the graphed results of these experiments. FIG. 1a shows that the addition of the peptide of SEQ ID NO.1 at 100 ng/ml to cells grown without PTH increases the levels of 1α-hydroxylase mRNA approximately 200%. The addition of the peptide of SEQ ID NO.1 at 500 ng/ml to cells grown without PTH increases the levels of 1α-hydroxylase mRNA approximately 500% (p<0.05). FIG. 1b shows that the peptide of SEQ ID NO.1 does not further increase the levels of 1α-hydroxylase mRNA when cells are grown in the presence of 100 nM PTH. The peptide of SEQ ID NO.1, therefore, is a potent inducer of 1α-hydroxylase gene expression. Since the 1α-hydroxylase gene encodes the final step in the biosynthetic pathway of calcitriol, any increase in its expression would be expected to increase the amount of 1α-hydroxylase enzyme and increase the rate of synthesis of calcitriol which would result in an increase in cellular and circulating levels of calcitriol.

### EXAMPLE 2

### Dentonin Does Not Require Protein Kinase A to Regulate 1α-Hydroxylase mRNA Expression

PTH and the peptide of SEQ ID NO.1 induce the expression of 1α-hydroxylase mRNA. CL-8 cells were grown with or without the peptide of SEQ ID NO.1, or with PTH, in the presence or absence of compound H-89, a protein kinase A inhibitor and the effects of H-89 on the induction of 1α-hydroxylase by the peptide of SEQ ID NO.1 of PTH were investigated. In the same experimental system as Example 1, at the point the cells were split, and in a first experiment, H-89 was not added to the cells, and PTH at a concentration of 100 nmol, or the peptide of SEQ ID NO.1 at concentrations of either 100 or 500 ng/ml were added to the cells. In a parallel second experiment, H-89 at a concentration of 10 uM was added to the cells with PTH at a concentration of 100 nmol, or the peptide of SEQ ID NO.1 at concentrations of either 100 or 500 ng/ml. Cells were grown for a further 5 hours, and RNA was harvested and expression levels of 1α-hydroxylase were determined in each sample using standard Northern blotting or RT-PCR procedures.

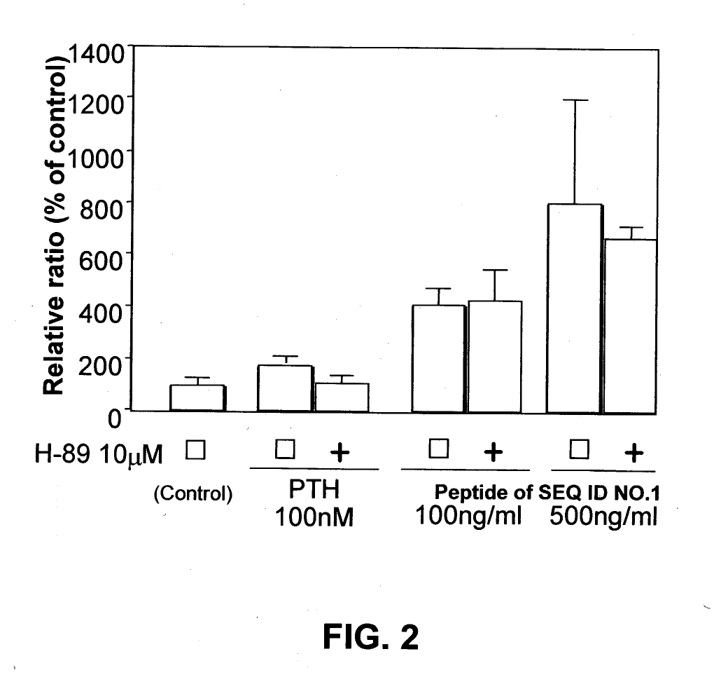

FIG. 2 shows the graphed results of these experiments. FIG. 2. shows that the peptide of SEQ ID NO.1 at concentrations of either 100 ng/ml or 500 ng/ml induced the expression levels of 1α-hydroxylase mRNA to significantly higher levels than PTH at a concentration of 100 nM. FIG. 2 also shows that H-89 inhibited the inducing effects of PTH on 1α-hydroxylase mRNA expression. Because H-89 is a potent and specific inhibitor to protein kinase A (PKA), it could therefore inhibit PTH induces 1α-hydroxylase mRNA expression through a PKA-dependent pathway.

In this experiment, H-89 did not inhibit the stimulatory effects of the peptide of SEQ ID NO.1 on 1α-hydroxylase mRNA expression. The peptide of SEQ ID NO.1, therefore induces 1α-hydroxylase through a PKA-independent pathway. The peptide of SEQ ID NO.1 therefore works in a different signal transduction pathway than PTH. PTH binds a PTH receptor (PTHR) on cell membrane of bone or renal tubule cells, and a signal is transduced to effect the expression of particular genes via a PKA-dependent pathway. PTH, and analogs of PTH, are frequently proposed as agents for the treatment of vitamin D metabolism-related disorders, such as osteoporosis (for example U.S. Pat. Nos. 6,316,410, 6,147,186 and 5,969,095). Although PTH and its analogs induce the expression of 1α-hydroxylase mRNA, and thereby increase active vitamin D synthesis in renal tubule cells, it simultaneously promotes calcium re-absorption in renal tubule cells that results in hypercalcemia. Thus, the effectiveness of PTH and its analogs is compromised by its effects that cause hypercalcemia. The peptide of SEQ ID NO.1, because it has been found to induce 1α-hydroxylase mRNA expression through a PKA-independent pathway, may not have the negative side-effects of PTH and it's analogs as a treatment for vitamin D-related diseases.

### EXAMPLE 3

### Dentonin Does Not Induce 24-Hydroxylase mRNA Expression

The 24-hydroxylase is an enzyme which places a hydroxylates 25-monohydroxyvitamin D3, the immediate precursor for calcitriol, or calcitriol itself. In the case of the precursor, hydroxylation at the 24 position causes the pathway to “bypass” the active form of the compound. Hydroxylation of active calcitriol inactivates the compound.

CL-8 cells were grown as above and the peptide of SEQ ID NO.1 was either not added to the cells, or added to the cells at a concentration of either 100 or 500 ng/ml. Cells were grown for a further 5 hours, and RNA was harvested using methods described above and expression levels of 24-hydroxylase were determined in each sample using methods described above.

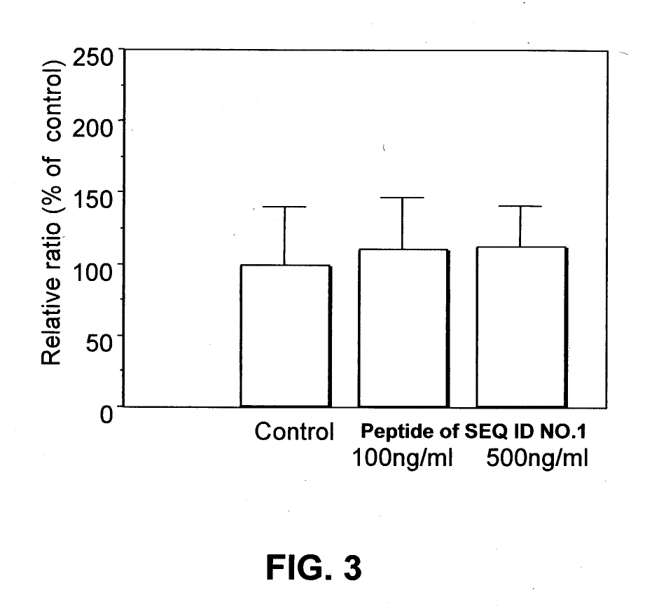

FIG. 3 shows the graphed results of this experiment. No significant induction of the 24-hydroxylase was observed with either 100 or 500 ng/ml of the peptide of SEQ ID NO.1. Thus, the peptide of SEQ ID NO.1 did not induce the expression of 24-hydroxylase. This result is significant because the peptide of SEQ ID NO.1 can increase the expression of the 1α-hydroxylase without increasing the expression of the 24-hydroxylase, thereby increasing the overall concentration of calcitriol in the cells and circulation.

### EXAMPLE 4

### Dentonin Does Not Induce 24-Hydroxylase mRNA Expression when in Combination with 1α,25-Dihydroxyvitiman D3

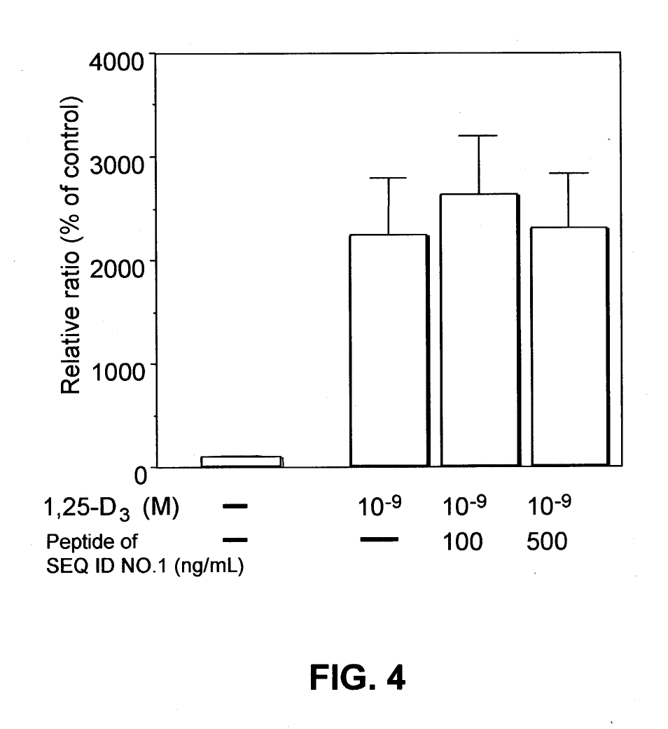

Calcitriol (1α,25 dihydroxyvitamin D3) is a known inducer of 24-hydroxylase mRNA expression. In order to determined the effects of the peptide of SEQ ID NO.1 on the 24-hydroxylase mRNA inducing effects of Calcitriol, CL-8 cells were grown as above, 1α,25-dihydroxyvitamin D3 at a concentration of 10⁻⁹ M was added to the cells and the peptide of SEQ ID NO.1 was either not added to the cells, or added to the cells at a concentration of either 100 or 500 ng/ml. Cells were grown for a further 5 hours, and RNA was harvested using methods described above and expression levels of 24-hydroxylase mRNA were determined in each sample using methods described above. As indicated in FIG. 4, a significant increase in the levels of 24-hydroxylase mRNA was observed on addition of 1α,25-dihydroxyvitamin D3. However, subsequent addition of the peptide of SEQ ID NO.1 at either 100 or 500 ng/ml had no effect on the expression levels of 24-hydroxylase mRNA. These results suggest that the peptide of SEQ ID NO.1 does not effect at all 24-hydroxylase mRNA expression with or without it induction by other mechanisms.

Further biologically active homologs, deletion and substitution variants of SEQ ID No. 1 have been identified, and the sequences are shown in SEQ ID NOS:2-18.

### EXAMPLE 5

### In vivo Effects of Dentonin Peptides

In order to study the in vivo effects of the dentonin peptide of SEQ ID No. 1, the serum 1α,25-dihydroxyvitamin D3 levels were studied after the subcutaneous injection of the peptide into the primates at different doses. The serum levels of the injected peptide were also examined.

The peptide of SEQ ID No. 1 was solubilized in carrier (0.9% sodium chloride) at concentrations to facilitate administration of doses of 1 and 50 mg/kg. Cynomolgus monkeys, approximately 2 years of age and weighing between approximately 1.5-2.5 kg were administered the peptide of SEQ ID No. 1 by subcutaneous injection. The peptide of SEQ ID No. 1 at different concentrations was administered at a constant volume of 1 ml/kg. Vehicle alone (0.9% Sodium Chloride) was administered in similar volumes at the same time points to act as a control.

Blood samples were collected at 2 and 24 hours post injection, and serum was prepared and stored in aliquots at −70° C. for further analysis. Serum was assayed for 1α,25-dihydroxyvitamin D3 using a validated radioimmunoassay (Quest Diagnostics). The LOQ for this assay was determined to be approximately 3 pg/ml.

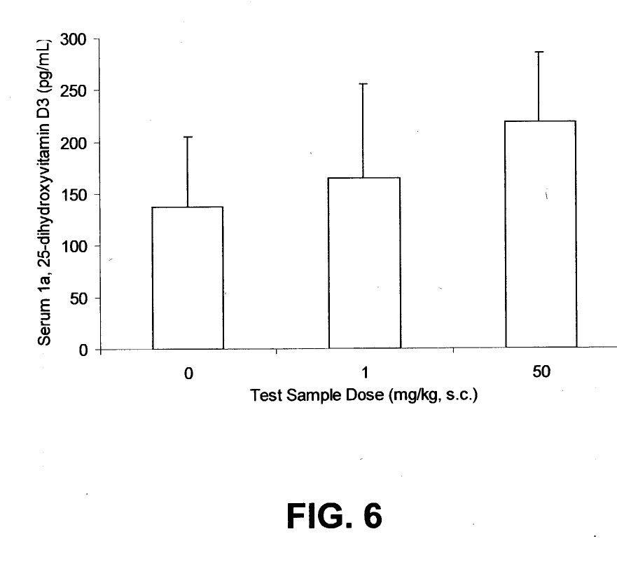

Data are shown in FIG. 6 as pg/ml 1α,25-dihydroxyvitamin D3 in serum. Data represent the mean and standard deviation of 8 animals (4 males and 4 females) for each dosing group.

As FIG. 6 demonstrates, the treatment of these animals with subcutaneously administered peptide of SEQ ID No. 1 increased the serum levels of 1α,25-dihydroxyvitamin D3, and raises the serum levels of 1α,25-dihydroxyvitamin D3 in a dose dependent manner. For example, 50 mg/kg group showed significantly higher value (p<0.05) as compared to 0 mg/kg (Vehicle) group.

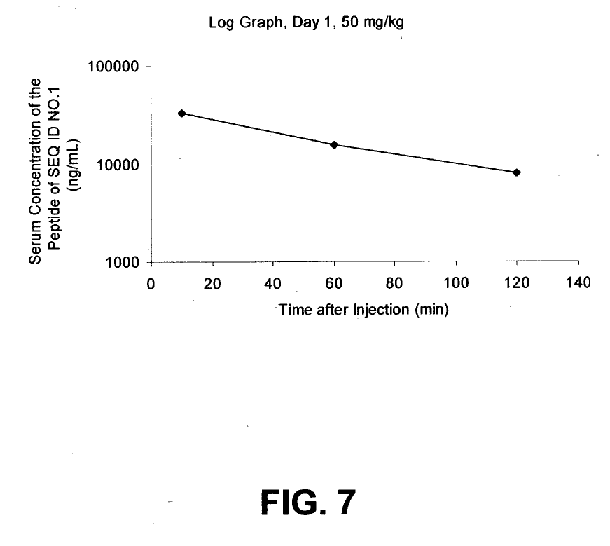

The serum concentration of the peptide of SEQ ID No. 1 was measured when 50 mg/kg of the sample was subcutaneously injected to the monkeys. As shown in FIG. 7, the serum concentration of the peptide of SEQ ID No. 1 after its administration was significantly higher than its biologically active concentration demonstrated in its in vitro experiment (see Example 1).

No adverse events were observed in any of the monkeys received the test peptide injection.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An isolated polypeptide comprising dentonin, wherein said polypeptide is characterized by a biological activity that induces a 25-hydroxyvitamin D3 1α-hydroxylase activity.

2. The isolated polypeptide of claim 1, wherein said dentonin is at least 50% identical to 15 contiguous amino acids of any one of SEQ ID NOS:1-18.

3. The isolated polypeptide of claim 1, wherein said dentonin comprises the sequence:
ArgX₁X₂AsnAspX₃X₄ProPheSerX₅X₆X₇Gln,
where X₁-X₇ are any single amino acids.

4. The isolated polypeptide of claim 1, wherein the polypeptide comprises at least 15 contiguous amino acids any one of SEQ ID NOS: 1-18.

5. The isolated polypeptide of claim 1, wherein the polypeptide comprises the amino acid sequence of SEQ ID NO: 1.

6. The isolated polypeptide of claim 1, wherein the polypeptide is further characterized by a biological activity that does not significantly induce vitamin D 24-hydroxylase activity.

7. The isolated polypeptide of claim 1, wherein the polypeptide is further characterized by a biological activity that increases calcitriol amounts.

8. An isolated nucleic acid encoding the polypeptide of claim 1.

9. A method for increasing 25-hydroxyvitamin D3 1α-hydroxylase activity in a cell, said method comprising:
contacting said cell with an isolated polypeptide of claim 1;
wherein 25-hydroxyvitamin D3 1α-hydroxylase activity is increased in said cell in comparison to a cell not contacted with the isolated polypeptide of claim 1.

10. The method of claim 9, wherein vitamin D 24-hydroxylase activity is not significantly induced in said cell.

11. The method of claim 9, wherein said cell is a cell of a mammalian host.

12. A formulation comprising:
a therapeutically effective amount of a polypeptide of claim 1.

13. The formulation of claim 12, further comprising a carrier.

14. The formulation of claim 13, wherein the carrier is a saline solution and the formulation is injectable.

15. A method for increasing levels of calcitriol in a subject, said method comprising:
administering to said subject a therapeutically effective amount of the formulation of claim 12;
to increase said levels of calcitriol.

16. A method for treatment of a subject in need of said treatment, said method comprising:
administering to said subject a therapeutically effective amount of the formulation of claim 12;
to treat said subject.

17. The method of claim 16, wherein said subject has a vitamin-D related condition.

18. The method of claim 17 wherein the condition is rickets, osteomalacia, osteoporosis, Paget's disease, osteopenia, osteosclerosis or renal osteodystrophy, psoriasis, medullary carcinoma, Alzheimer's disease, hyperparathyroidism, hypoparathyroidism, pseudoparathyroidism, secondary parathyroidism, diabetes, cirrhosis, obstructive jaundice or drug-induced metabolism, glucocorticoid antagonism, hypercalcemia, malabsorption syndrome, steatorrhea, chronic renal disease, hypophosphatemic vitamin D-resistant rickets, vitamin D-dependent rickets, rickets type I, rickets type II sarcoidosis, leukemia, prostate cancer, breast cancer, colon cancer, organ transplantation, bone fracture, undecalcified teath or an immunodisorder.

19. The method of claim 18 wherein the disease is osteoporosis, Paget's disease, osteomalacia, rickets, renal osteodystrophy, hyperparathyroidism, hypercalcemia, rickets type I, rickets type II and bone fracture.

20. A system for increasing target nucleic acid expression in a cell, said system comprising:
(a) isolated dentonin polypeptide; and
(b) a 25-hydroxyvitamin D3 1α-hydroxylase promoter operably linked to said target nucleic acid,
wherein said dentonin induces the activity of said 1α-hydroxylase promoter to increase the expression of said target nucleic acid.

21. A method of increasing the expression of a target nucleic acid in a cell, said method comprising:
operably linking a target nucleic acid to a 1α-hydroxylase promoter to form an expression cassette;
transferring said expression cassette into a cell; and
contacting the cell with the isolated polypeptide of claim 1,
to increase expression of said target nucleic acid.

22. The method of claim 21 wherein the cell is a kidney cell.


SEQUENCE LISTING (23 sequences)

SEQ ID NO: 1 - protein, 23 aa, Homo sapiens
  Synthetic oligopeptide
TDLQERGDNDISPFSGDGQPFKD

SEQ ID NO: 2 - protein, 23 aa, Artificial sequence
  Synthetic oligopeptide
TDLQERDGNDISPFSGDGQPFKD

SEQ ID NO: 3 - protein, 23 aa, Artificial sequence
  Synthetic oligopeptide
TDLQERGDNDISPFSDGDQPFKD

SEQ ID NO: 4 - protein, 23 aa, Artificial sequence
  Synthetic oligopeptide
TDLQERGDNDMSPFSGDGQPFKD

SEQ ID NO: 5 - protein, 23 aa, Artificial sequence
  Synthetic oligopeptide
PDLLVRGDNDVPPFSGDGQHFMH

SEQ ID NO: 6 - protein, 21 aa, Artificial sequence
  Synthetic oligopeptide
TDLQERGDNDISPFSGDGQPF

SEQ ID NO: 7 - protein, 19 aa, Artificial sequence
  Synthetic oligopeptide
TDLQERGDNDISPFSGDGQ

SEQ ID NO: 8 - protein, 20 aa, Artificial sequence
  Synthetic oligopeptide
DLQERGDNDISPFSGDGQPF

SEQ ID NO: 9 - protein, 19 aa, Artificial sequence
  Synthetic oligopeptide
ERGDNDISPFSGDGQPFKD

SEQ ID NO: 10 - protein, 15 aa, Artificial sequence
  Synthetic oligopeptide
ERGDNDISPFSGDGQ

SEQ ID NO: 11 - protein, 22 aa, Artificial sequence
  Synthetic oligopeptide
DLQERDGNDISPFSGDGQPFKD

SEQ ID NO: 12 - protein, 21 aa, Artificial sequence
  Synthetic oligopeptide
TDLQERDGNDISPFSGDGQPF

SEQ ID NO: 13 - protein, 20 aa, Artificial sequence
  Synthetic oligopeptide
DLQERDGNDISPFSGDGQPF

SEQ ID NO: 14 - protein, 15 aa, Artificial sequence
  Synthetic oligopeptide
ERDGNDISPFSGDGQ

SEQ ID NO: 15 - protein, 15 aa, Artificial sequence
  Synthetic oligopeptide
ERGDNDMSPFSGDGQ

SEQ ID NO: 16 - protein, 15 aa, Artificial sequence
  Synthetic oligopeptide
VRGDNDVPPFSGDGQ

SEQ ID NO: 17 - protein, 24 aa, Artificial Sequence
  Synthetic oligopeptide
CTDLQERGDNDISPFSGDGQPFKD

SEQ ID NO: 18 - protein, 24 aa, Artificial Sequence
  Synthetic oligopeptide
TDLQERGDNDISPFSGDGQPFKDC

SEQ ID NO: 19 - DNA, 6017 bp, Homo sapiens
  Synthetic oligopeptide
gaattcctgaggatgggcctaaaggggctgggctcactggtagaagtgggaatatcagagactgactagtgtagcttggtcacctagtccctactaaaaagccttatagcctttcctaggatgagactttgaggctcctagacaaaggcactctcccaggaggaaaatcttaggccctccctcccatgagggtcattgcaacatgagacccaagggagttgtgaagtcagccccagccccgcctactgttcctgggtgctaatccccagcacagaccactcaggaggagggattggctgaggagcttggagagggggcgtcatcacctcacccaaaggttaaataggggttgagatatgatgctcaggagaagcgctttctttcgcgagcaccctgaaccagaccatgacccagaccctcaagtacgcctccagagtgttccatcgcgtccgctgggcgcccgagttgggcgcctccctaggctaccgagagtaccactcagcacgccggagcttggcagacatcccaggcccctctacgcccagctttctggccgaacttttctgcaagggggggctgtcgaggctacacgagctgcaggtaggaagggacgcctttcccgagacagagtgctggggaaactggttttgacagcgtcagaaaggactgactagtgcagaccaaatgtgggacagccagagagaacggatgcccatgaaataaggaaaaggcgagttgaggctgggggcggtgtggctacactcgggcagagcccgtcccgactcttatcagagggcgctgcgaaagcgccttctcgctgtcctgaggtgtggagatcctgcagataaagtacaactgcgcgggagggggaggccggagtgggcagtaccctccgcctgcttgcggcttaaagctacatgggttccttttcattcactgaggactcgtcctgagatggacagtccagacatggaacttttagagatttctcctcgaccgagatgatcagagaggtcctgaatgtctgccttgcacaaagttccggttttgcccatcacactcaaactttctagggatgggaaatgagagaggaggagtttgcagtatcaaaggcatacactagttgggcacccgagaaagagcagaagctccctattcccaagcccagtcaacgcccttggcgtgggcacaggtcaagctgaaagtccccgcccaggtatccaagtgtccgctgtgtccgctcccccaggtgcagggcgccgcgcacttcgggccggtgtggctagccagctttgggacagtgcgcaccgtgtacgtggctgcccctgcactcgtcgaggagctgctgcgacaggagggaccccggcccgagcgctgcagcttctcgccctggacggagcaccgccgctgccgccagcgggcttgcggactgctcactgcgttgagtcttctctgcccccagaagcccagacgccctgcaccggccctctcctgctgcgggtcccgaaactatcaatctgggggcatggtgggaggtcggctgtcccactctaccattgggcatcttggggttcccatcccagtctgcttcacccacactcaggttgagccccaacctcactccccgcctacctccttatcctttgaggcccgaatcctgggaactgggggaaccggaggaattgggctagggtacaaggatctggatggaatgaatgtaaggttctgtgactcaagttacccgggaaccccggcccctgagggcaggaaatgagtaaagaggaagggggctggaagacaggcgcaggccggtgcaggtttccgtaccccaagggggcagacgcaatccctctcctggccaccgcagccgaacgcgctccttcactgcagccagtcccgtcgagccgtccccaccttcccgatgcgcactctctcctcaaccctgcagggaaggcgaagaatggcaaaggctccgcagtctcctggccccgctcctcctccggcctcaagcggccgcccgctacgccggaaccctgaacaacgtagtctgcgaccttgtgcggcgtctgaggcgccagcggggacgtggcacggggccgcccgccctggttcgggacgtggcgggggaattttacaagttcggactggaaggtgagtcccaggacagagctgggcaggcgtcgggggcgccctaccagagcctcccggaaccctgacggcgccccctcccgacgaggcatcgccgcggttctgctcggctcgcgcttgggctgcctggaggctcaagtgccacccgacacggagaccttcatccgcgctgtgggctcggtgtttgtgtccacgctgttgaccatggcgatgccccactggctgcgccaccttgtgcctgggccctggggccgcctctgccgagactgggaccagatgtttgcatttggtaaggcacaggtcgaggtggaaatgggggaatgtaaagctgtccaggggtagcgaggtattcacgtgccttctacccacgcagctcagaggcacgtggagcggcgagaggcagaggcagccatgaggaacggaggacagcccgagaaggacctggagtctggggcgcacctgacccacttcctgttccgggaagagttgcctgcccagtccatcctgggaaatgtgacagagttgctattggcgggagtggacacggtgaggttctccctccgtgctgtgagccggttccagggcttagcctccgcagactccggctccatttttctgttgcaggggatccattatggccacgtagaccagcttggcttagcaccctgtagccccagactcttccataatctgcaccctctgctgggttctcacacccaacacctctcttgctttcacatgtttttcaggtgtccaacacgctctcttgggctctgtatgagctctcccggcaccccgaagtccagacagcactccactcagagatcacagctgccctgagccctggctccagtgcctacccctcagccactgttctgtcccagctgcccctgctgaaggcggtggtcaaggaagtgctaaggtgagggggaaggagaggaggaacaagaggaaatgccaaggaagggctggggaagcaactagtggatggaagcagggagatagcagagaaaaatggccctctactcctggccaaaaagggtttggaagttggaaacaatgagaagggggctgcagcccctagcctcatcttgttgtctccattttgtgctttgcaacctagactgtaccctgtggtacctggaaattctcgtgtcccagacaaagacattcatgtgggtgactatattatccccaaaaatgtgagtaaagcctatgcaccctttctactgagccattcctcactatcttgggccccaaccctgttctcaaacactctcctaaaagcccttcaaacactccccttgacccctctctaggatatgcctttctaggatgtaatggaacagtggggagatccttccaaaagtagccccagaaaacttccccatcacccaccaggacctgcctttgtccctgtctgaatatcctcttcttcatgcctgccctattctgagcccaaactgacaagtttgttttcctttgcaccagacgctggtcactctgtgtcactatgccacttcaagggaccctgcccagttcccagagccaaattcttttcgtccagctcgctggctgggggagggtcccaccccccacccatttgcatctcttccctttggctttggcaagcgcagctgtatggggagacgcctggcagagcttgaattgcaaatggctttggcccaggtgagtgctctagattttataccttccccagactggagagaccctaaccctctaaagttgtgagctctttcccctgacaagcataggaaatcatataagacctggtagaatgaatcttctgaaatatgataagcccattataggcctggagtgtaagtgagggtattcaaactattttttccctaccataatccctcacccttattaaccaagaagtcccctactggccacaggtgccacccaatcattgaccattctaacactaataatgcatgccctttaccaattggattaccaatgaatccccccattatagatatctttcatagtaatgctcaccttcttccctttccagatcctaacacattttgaggtgcagcctgagccaggtgcggccccagttagacccaagacccggactgtcctggtacctgaaaggagcatcaacctacagtttttggacagatagtcccatggaaagagactgtcatcatcaccctttcattcatcatagggataagattttttgtaggcacaagaccaaggtatacatcttcccctaatgcctatctgaccaaactggatagaaccaccatagtgaagtgtgaggcggccctgaccaatgtgtgaagtatgcacttggcctgactcaggaagccaggtgagaaaaccatggtctctctgcttgcttggcccttctgatcatgtatgcatcccccaaggatgaaatcagattttaactaataatgctggatggcctgaggaaagattcaactgcctctctttttgggctttcatagtgttcattgatgctgctggctaagcatttatcaaagcataagctcagtaactgtgcatctggtctgtacctggttggtccttcgtctttgcatgtaagctctttgagaggaagggtgaagccttatttgttttttatgtcccctgccagggcctgtctctgactaggtgtcaccatacacattcttagattgaatctgaaccatgtggcagaagggataagcagcttacttagtaggctctgtctacccccttccttctttgtcttgcccctaggaaggtgaatctgccctagcctggtttacggtttcttataactctcctttgctctctggccactattaagtgggtttgccccatcacttagttctcaggcagagacatctttgggcctgtccctgcccaggcctctggctttttatattgaaaatttttaaatattcacaaattttagaataaatcaaatattccattcttgattttgacttatttctctcatattcccacttcctacaagacctggaggcaagactctaaccctcccatctcccaccttggtttctcaggtcctgggggtgacccccataacaatgggagtctggacgcctggaagagtagtttgtaacacaaacccagacatgtttctgtctgtagtgggaacatccaagtctagtctggcttcctgtaagagattaagggggtggggggtgaaaagtaacggcaggggctaagctgtaaaatgggcccggagctagggatttgcagacatgggcagaggttgctgactctggtaaggaaagtgcttttggactgccaggattgaaggaagtcctagattggtcactttgccctctctggaaaagttgttccaagataatttccttcagaggaagggatacaagcagagaagtggaatcagacagacagaatgaaagctgagtgtgggggtggccagaagccctggggcactgaggcaggataagggcggtgggcagagggagactgtttccctaagtcagcattctctggccccccagaggctgggcacagctctcctcttgtgatgtatacagattaactcatcttccatccaaataaatctctcagcacactgcacagttccttctgcatcccagattacccttcccatgtcaggacttgccccccttcccctaccgctcaccctattccaattagctgggtgtagatggagatttcacttcacacttcttcctcctccacaccttggcctcctggcctagacatggctccctgaccgcagaaactccagtagccaccaggccttttgcaggggcatctaattatgcaagatctacagaggggcagggcagacccaaagcctcgtacaaagatccgaattc

SEQ ID NO: 20 - DNA, 20 bp, Artificial Sequence
  Synthetic oligonucleotide
actgtaccctgtggtacctg

SEQ ID NO: 21 - DNA, 20 bp, Artificial sequence
  Synthetic oligonucleotide
ccttgaagtggcatagtgac

SEQ ID NO: 22 - DNA, 22 bp, Artificial sequence
  Synthetic oligonucleotide
aggcccagagcaagagaggtat

SEQ ID NO: 23 - DNA, 22 bp, Artificial sequence
  Synthetic oligonucleotide
catgtcgtcccagttggtaaca